(19) United States
(12) Patent Application Publication
(10) Pub. No.: US 2014/0093460 A1
(43) Pub. Date: Apr. 3, 2014

(54) NAIL ENAMEL COMPOSITION

(71) Applicant: Amy Doan, Garden Grove, CA (US)

(72) Inventor: Amy Doan, Garden Grove, CA (US)

(21) Appl. No.: 14/029,582

(22) Filed: Sep. 17, 2013

Related U.S. Application Data
(63) Continuation-in-part of application No. 13/633,439, filed on Oct. 2, 2012.

(51) Int. Cl. A61K 8/40; A61Q 3/02

(52) U.S. Cl. CPC A61K 8/40; A61Q 3/02
USPC 424/61

(57) ABSTRACT

A nail enamel composition has a film-forming mixture and an accelerator mixture. The film-forming mixture includes 9-28% nitrocellulose, by weight, in a suitable solvent. The accelerator mixture includes acetone and/or ethyl acetate. The composition further includes less than 1% cyanoacrylate, by weight. The nail enamel composition is substantially free of graphite fibers.

### CROSS-REFERENCE TO RELATED APPLICATIONS

This application for a utility patent is a continuation-in-part of a previously filed utility patent, now abandoned, having the application Ser. No. 13/633,439, filed Oct. 2, 2012.

### BACKGROUND OF THE INVENTION

1. Field of the Invention

This invention relates generally to nail enamels, and more particularly to a nail enamel composition that includes cyanoacrylate to provide improved drying times.

2. Description of Related Art

For purposes of this application, the term “nail enamel” is hereby defined to include any form of film-forming composition such as enamels, lacquers, top coats, base coats, and similar or equivalent materials that are used to coat a nail, whether clear or pigmented. For purposes of this application, the term “nail” is hereby defined to include natural fingernails, toenails, as well as artificial nails, nail tops, or plastic nails.

Conventional nail enamel products (i.e., nail polish) typically contain nitrocellulose as the main film-forming resin, and may further include additional resins, plasticizers, and other agents. Such products typically also include pigments, and a thixotropic agent for suspending the pigment, as well solvents such as n-butyl acetate, ethyl acetate, and toluene.

Cooper et al., U.S. Pat. No. 4,646,765 teaches a nail composition that includes a combination of cyanoacrylate and graphite fibers. The cyanoacrylate bonds the graphite fibers to the nail for strengthening the nail. While Cooper teaches the use of cyanoacrylate in conjunction with graphite fibers, there is no teaching in Cooper to add cyanoacrylate in regular nail polish that does not include graphite fibers.

Srulevitch et al., US 2008/0112908, teaches a nail fungal treatment that includes nitrocellulose. However, there is no teaching in Srulevitch to include cyanoacrylate.

Galante, U.S. Pat. No. 4,384,058, teaches such an nail enamel, and further teaches the inclusion of an auxiliary resin to strengthen the enamel, preferably a film-forming styrene-acrylonitrile-acrylic terpolymer; however, a film-forming alkyl cyanoacrylate is also listed as an option for strengthening the enamel. The alkyl cyanoacrylate is present in the amount of 2-12%, and is preferably 5% of the enamel. Also, such cyanoacrylate additives typically include toluene, which is a disadvantage due to the offensive odor. Furthermore, it is noted in the discussion that their formulation does not provide a suitable gloss.

While nail polish containing nitrocellulose is known, it is not known to add cyanoacrylate to such a nail polish, except, as in Cooper, it is added in conjunction with graphite fibers for bonding the fibers to the nail, or in Galante, where it is added in much larger quantities. The prior art does not teach a nail polish that includes less than 1% cyanoacrylate, and that does not include graphite fibers.

### SUMMARY OF THE INVENTION

The present invention teaches certain benefits in construction and use which give rise to the objectives described below.

The present invention provides a nail enamel composition comprising a film-forming mixture that includes 9-28% nitrocellulose, by weight, in a suitable solvent, and an accelerator mixture. The accelerator mixture includes acetone and/or ethyl acetate. The composition further includes less than 1% cyanoacrylate, by weight. The nail enamel composition is substantially free of graphite fibers.

A primary objective of the present invention is to provide a nail enamel composition having advantages not taught by the prior art.

Another objective is to provide a nail enamel composition that dries more quickly.

Other features and advantages of the present invention will become apparent from the following more detailed description, taken in conjunction with the accompanying drawings, which illustrate, by way of example, the principles of the invention.

### BRIEF DESCRIPTION OF THE DRAWINGS

The accompanying drawing illustrates the present invention. In such drawing:

FIG. 1 is a flow diagram illustrating a method of preparation of a nail enamel composition according to one embodiment of the present invention.

### DETAILED DESCRIPTION OF THE INVENTION

The above-described drawing figures illustrate the invention, a nail enamel composition and a method of preparing the nail enamel composition for use on a nail of a user.

The nail enamel composition comprises a film-forming mixture adapted for application to a human nail, and a cyanoacrylate mixture. The method of mixing the nail enamel composition is discussed in greater detail below.

The film-forming mixture includes nitrocellulose as a major component, and a compatible solvent. As discussed in greater detail below, the film-forming mixture may include nitrocellulose, ethyl and/or butyl acetate, isopropanol, and a free radical inhibitor. In one embodiment, the film-forming mixture includes triphenyl phosphate, and may further include additional chemicals, as discussed in greater detail below.

The cyanoacrylate mixture comprises cyanoacrylate and an accelerator mixture, and may further include a free radical inhibitor that does not include toluene, such as butylated hydroxyanisole. The accelerator mixture may include acetone and ethyl acetate, as discussed in greater detail below. The cyanoacrylate may first be mixed with the accelerator mixture, as discussed below, prior to its addition to the film-forming mixture, to prevent crystallization of the mixture.

The film-forming mixture may comprise nitrocellulose as a major component (i.e., enough so that the mixture functions as a nail polish), and may include any of the number of additives typically found in nail enamel (i.e., nail polish). In an alternative embodiment, the film-forming mixture may comprise a top coat, or similar coating applied on top of the nail enamel. In some embodiments, the film-forming mixture may include color pigment, and/or any form of texturing or decorative colors or features known in the art.

In one embodiment, the cyanoacrylate is 2-Ethyl cyanoacrylate. In this embodiment, the free radical inhibitor may be butylated hydroxyanisole. The solvent may be ethyl acetate and/or butyl acetate, and/or other acceptable solvents known in the art. In one embodiment, the cyanoacrylate mixture is substantially free of toluene. For purposes of this application, the term “substantially free of toluene” is defined to mean either no toluene at all, or such small traces of toluene that it does not cause any negative side effects such as irritation of the skin, noxious smell, or any other negative effects of toluene.

In one embodiment, the nail enamel composition comprises the following:
45-67% ethyl acetate and/or butyl acetate;
9-19% isopropanol;
9-28% nitrocellulose;
1-9% triphenyl phosphate;
0.01-1% diacetone alcohol;
0.01-0.2% butylated hydroxyanisole;
an operative amount of cyanoacrylate for substantially speeding the drying of the nail enamel composition, less than 1% of the total weight of the nail enamel composition. Each percentage is a percentage by weight.

In one embodiment, the nail enamel composition comprises 27-37% ethyl acetate and 18-28% butyl acetate. In one embodiment, the nail enamel composition comprises 0.05-1% cyanoacrylate.

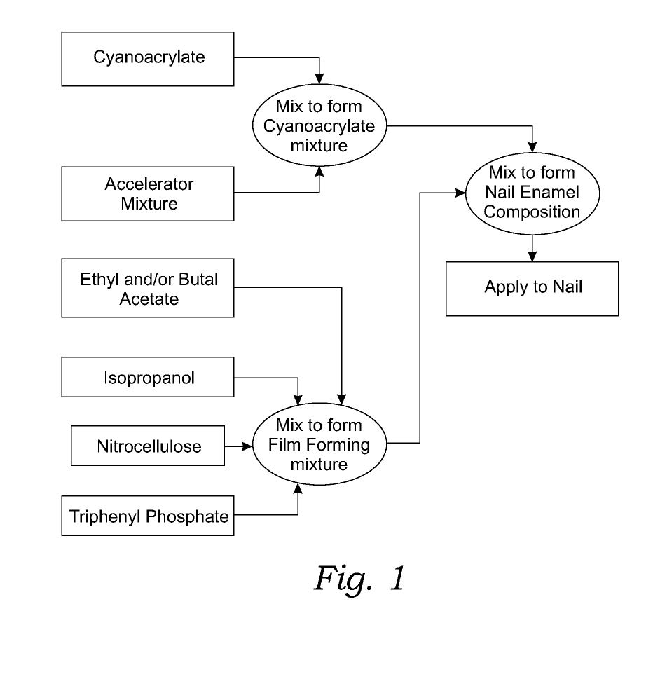

FIG. 1 is a flow diagram illustrating a method of preparing the nail enamel composition according to one embodiment of the present invention. As illustrated in FIG. 1, the method for forming the nail enamel composition comprises the steps of first mixing the cyanoacrylate with the accelerator mixture, to form the cyanoacrylate mixture. The cyanoacrylate may first be mixed with the accelerator mixture, as illustrated, to prevent crystallizing of the nail enamel composition. The cyanoacrylate mixture may further include a free radical inhibitor, in this case butylated hydroxyanisole, to form a cyanoacrylate mixture. In one embodiment, 0.01-3% of the cyanoacrylate mixture is the cyanoacrylate, once mixed with the accelerator mixture. In another embodiment, 0.1-5% of the cyanoacrylate mixture is the cyanoacrylate, once mixed with the accelerator mixture. In yet another embodiment, the cyanoacrylate mixture includes about 1% cyanoacrylate (about being defined to mean +/−10 percent). The cyanoacrylate mixture may further include less than 0.15% of the butylated hydroxyanisole, such as approximately 0.1% of the butylated hydroxyanisole.

The accelerator mixture may include 50-70% acetone and 30-50% ethyl acetate. Obviously, those skilled in the art may devise alternative formulations, and may add additional chemicals that don't interfere with the function of the accelerator mixture, and such alternative formulations should be considered within the scope of the present invention.

Separately, the nitrocellulose mixture is formed by mixing a film-forming mixture comprising nitrocellulose as a major component, with a compatible solvent to form the nitrocellulose mixture. The nitrocellulose mixture is then mixed with the cyanoacrylate mixture to form the nail enamel composition that is substantially free of toluene.

Keeping the cyanoacrylate, mixed with the free radical inhibitor, separate from the film-forming mixture extends the shelf life of the cyanoacrylate, so it is helpful to maintain the cyanoacrylate mixture separately from the film-forming mixture until they are ready to be mixed, packaged, and sold.

While FIG. 1 illustrates one embodiment of how the composition may be formed, those skilled in the art may devise alternative embodiments, and these alternative or equivalent are considered within the scope of the present invention.

The invention further includes a method of applying the nail enamel composition to a nail of a person or animal. In a first embodiment, a first layer of a nail bonding/preparation solution is applied. The nail bonding/preparation solution may include a primer, a bonding agent, and/or any other form of preparation solution known in the art. In another embodiment, however, this step may be omitted, depending upon the needs of the user.

A coat of nail base may then be provided. The nail base provides a protective layer that maximizes color adhesion and eliminates staining The nail base may comprise, in one embodiment, isopropyl alcohol, ethyl and butyl acetate, polyvinyl butyral, nitrocellulose, trimethyl pentanyl diisobutyrate, tosylamide formaldehyde resin, and benzophenone. In other embodiments, other known nail base formulations may be used.

One or more coats of the nail enamel composition are then applied, depending upon how dark the color is desired. Since the application of the nail enamel composition is well known in the art, and is described in greater detail above, further discussion of this step is not required.

Finally, a top coat may be applied. The top coat provides a glossy finish and may also include a wide range of special effects. The top coat may comprise standard ingredients, such as nitrocellulose, ethyl an butyl acetate, cellulose acetate butyrate, n-butyl alcohol, propyl acetate, trimethyl pentanyl diisobutyrate, phthalic anhydride/trimellitic anhydride/glycols copolymer, acrylates copolymer, acrylates copolymer, bis-phenylpropyl dimethicone, etocrylene, and isopropyl alcohol. These ingredients may be modified or substituted according to the requirements of one skilled in the art, and such alternatives should be considered within the scope of the present invention.

In another embodiment, the top coat may include various natural extracts, such as chamomilla recutita (matricaria) extract, citrus aurantium dulcis (orange) flower water, hydrolyzed conchiolin protein, lilium candidum bulb extract, retinyl palmitate, simmondsia chinensis (jojoba) seed oil, tocopherol, arachis hypogaea (peanut) oil, benzyl benzoate, butylene glycol, as well as acetyl tributyl citrate, sucrose benzoate, isopropyl alcohol, methylparaben, alpha-isomethylionone, and butylphenyl methylpropional.

The nail enamel composition is substantially free of graphite fibers. Prior art formulations described above include graphite fibers in combination with the cyanoacrylate, for the purpose of strengthening the nail. In this composition, the cyanoacrylate and the graphite fibers together form a strengthening structure. In the current invention, it is desirable that the nail enamel composition be “substantially free” of the graphite fibers, meaning that the nail enamel composition is not materially altered by the graphite fibers, excluding de minimis introductions of graphite fiber that have no impact on the final nail enamel composition.

As used in this application, the words “a,” “an,” and “one” are defined to include one or more of the referenced item unless specifically stated otherwise. Also, the terms “have,” “include,” “contain,” and similar terms are defined to mean “comprising” unless specifically stated otherwise. Furthermore, the terminology used in the specification provided above is hereby defined to include similar and/or equivalent terms, and/or alternative embodiments that would be considered obvious to one skilled in the art given the teachings of the present patent application.

What is claimed is:

1. A nail enamel composition comprising:
a film-forming mixture that includes 9-28% nitrocellulose, by weight, in a suitable solvent;
an accelerator mixture;
less than 1% cyanoacrylate, by weight; and
wherein the nail enamel composition is substantially free of graphite fibers.

2. The nail enamel composition of claim 1, further comprising 1-9% triphenyl phosphate, by weight.

3. The nail enamel composition of claim 1, further comprising a free radical inhibitor that does not include toluene.

4. The nail enamel composition of claim 3, wherein the free radical inhibitor is butylated hydroxyanisole.

5. The nail enamel composition of claim 1, further comprising:
45-67% ethyl acetate and/or butyl acetate;
9-19% isopropanol; and
1-9% triphenyl phosphate.

6. The nail enamel composition of claim 1, wherein the nail enamel composition further comprises: 27-37% ethyl acetate and 18-28% butyl acetate.

7. The nail enamel composition of claim 1, wherein the nail enamel composition further comprises a color pigment and a thixotropic agent for suspending the color pigment, and wherein the accelerator mixture includes acetone and/or ethyl acetate.

8. The nail enamel composition of claim 1, wherein the nail enamel composition is free of toluene.

9. A nail enamel composition comprising:
a film-forming mixture that includes 9-28% nitrocellulose, by weight, in a suitable solvent;
an accelerator mixture that includes acetone and/or ethyl acetate;
less than 1% cyanoacrylate, by weight; and
wherein the nail enamel composition is free of graphite fibers and toluene.

10. The nail enamel composition of claim 9, further comprising 1-9% triphenyl phosphate, by weight.

11. The nail enamel composition of claim 9, further comprising a free radical inhibitor that does not include toluene.

12. The nail enamel composition of claim 11, wherein the free radical inhibitor is butylated hydroxyanisole.

13. The nail enamel composition of claim 9, further comprising:
45-67% ethyl acetate and/or butyl acetate;
9-19% isopropanol; and
1-9% triphenyl phosphate.

14. The nail enamel composition of claim 9, wherein the nail enamel composition further comprises: 27-37% ethyl acetate and 18-28% butyl acetate.

15. The nail enamel composition of claim 9, wherein the nail enamel composition further comprises a color pigment and a thixotropic agent for suspending the color pigment.

16. A nail enamel composition consisting of:
45-67% ethyl acetate and/or butyl acetate;
9-19% isopropanol;
9-28% nitrocellulose;
1-9% triphenyl phosphate;
0.01-1% diacetone alcohol;
a thixotropic agent;
a color pigment;
0.01-0.2% butylated hydroxyanisole; and
less than 1% cyanoacrylate, by weight.
